(19) United States
(12) Patent Application Publication
(10) Pub. No.: US 2021/0177893 A1
(43) Pub. Date: Jun. 17, 2021

(54) Composition and method for treating and healing Medication-Related Osteonecrosis of the Jaw (MRONJ)

(71) Applicant: Chun Nan Lin, Rosemead, CA (US)

(72) Inventor: Chun Nan Lin, Rosemead, CA (US)

(21) Appl. No.: 17/110,249

(22) Filed: Dec. 2, 2020

Related U.S. Application Data
(60) Provisional application No. 62/946,626, filed on Dec. 11, 2019.

(51) Int. Cl. A61K 33/24; A61K 9/06; A61K 47/38; A61K 47/10; A61P 19/08

(52) U.S. Cl. CPC A61K 33/24; A61K 9/06; A61P 19/08; A61K 47/10; A61K 47/38

(57) ABSTRACT

A composition and method of preventing and treating all forms of osteonecrosis of the jaw are disclosed. The composition is comprised of 0.005 %-1.5% weight/volume (w/v) stannous salt, such as stannous fluoride, stannous chloride, or similar and may take the form of gel or similar. The prevention and treatment includes the application of the composition in the oral cavity and other body areas affected by Medication-Related Osteonecrosis of the Jaw (MRONJ).

### CROSS REFERENCE TO RELATED APPLICATIONS

The present application is continuing of an application entitled “Method and Composition for Preventing and Healing Osteonecrosis of the Jaw”, filed Dec. 11, 2019 and assigned Ser. No. 62/946,626.

### BACKGROUND OF THE INVENTION

### Field of the Invention

The composition is comprised of a 0.005%-1.5% weight/volume (w/v) stannous fluoride (stannous fluoride, stannous chloride, and stannous salts or similar) and may take the form of an oral gel or similar, to facilitate the healing and treating of osteonecrosis of the jaw symptoms (including but not limited to necrotic bone lesions, inflammation, fistula, and infection). The composition is applied in the oral cavity affected by medication-related osteonecrosis of the jaw (MRONJ) to produce an antimicrobial effect, sanitize, debride, and penetrate, eliminate and control biofilms associated with medication-related osteonecrosis of the jaw (MRONJ), to facilitate the healing of medication-related osteonecrosis of the jaw symptoms (MRONJ) and prevent the development of medication-related osteonecrosis of the jaw (MRONJ) by poor oral hygiene, traumatic, periodontitis, and xerostomia (dry mouth). The composition may be applied two to four times daily or continuously as a treatment or prevention regimen for medication-related osteonecrosis of the jaw (MRONJ).

### Description of Related Prior Art

Medication-related osteonecrosis of the jaw (MRONJ) is a well-known rare disease, but severe adverse effect of certain drugs, of which bisphosphonates are the most widely known clinical problem. MRONJ is defined by (1) current or previous treatment with a bisphosphonates or angiogenesis inhibitor, (2) exposed bone or bone that can be probed through an intraoral or extraoral fistula in the maxillofacial region and that has persisted for longer than 8 weeks, and (3) no history of radiation therapy to the jaws or metastatic disease to the jaws. (AAMOS, 2007) (4) Pain, soft-tissue edema and local infection or abscess formation with development of fistulas are features of MRONJ. When bone exposed in oral cavity, loosening of teeth may occur.

Medication-related osteonecrosis of the jaw (MRONJ) are believed to develop due to either 1) decreased blood flow to bone which occurs as a result of a traumatic event such as sites of previous tooth extraction or areas of trauma, or 2) non-traumatic factors. While the pathophysiology of medication-related osteonecrosis of the jaw (MRONJ) has not been well determined currently, some risk factors may potentially contribute to its development such as dentoalveolar surgery (tooth extraction, periapical surgery, dental implant, and periodontal surgery), treatment with antiangiogenic agents, therapy duration, concomitant steroid usage, concomitant oral disease, bisphosphonate use, high dose and/or prolonged use of antiresorptive agents (more than 2 years), a compromised immune system, denture wearing, diabetes, chemotherapy, age older than 65 years, periodontitis, poor oral hygiene, corticosteroid use, smoking, malignant disease (multiple myeloma, and breast, prostate, and lung cancer), and tissue trauma. However, these factors might contribute to the development of osteonecrosis is also unknown and still under investigation (AAMOS, 2007; Mariotti, 2008; Bejar et al., 2005). In a study by Marx et al., 76 consecutive patients referred to the University of Miami, Fla., USA, were reviewed in addition to 43 patients from other centers. Approximately a third had presented with asymptomatic exposed bone, which had been self-identified or noted on routine examination. Approximately two-thirds of patients presented with exposed bone in the presence of pain (Bamias A, Kastritis E, Bamia C et al. 2005). The reported incidence of MRONJ varies, but it is generally considered to be between 1% and 10% of patients taking IV bisphosphonates for the management of bone metastatic disease and between 0.001% and 0.01% in patients taking oral bisphosphonates for the management of osteoporosis (Yamashita J, McCauley L K).

Bisphosphonates are a class of drugs prescribed for diseases that prevent the loss of bone mass involving deleterious bone resorption, such as osteoporosis, malignant bone diseases in several forms of cancer (breast cancer and prostate cancer), Paget's disease, and other conditions that lead to bone fragility. In general, these drugs can be administered orally, intravenously, or parenterally and bind tightly to the surface of the bone surface directly beneath the bone cell which known as osteoclasts in bone to inhibit and disrupt the bone resorption activity. The resulting function depends on the specific chemical structure of the bisphosphonate compound. Bisphosphonates are inorganic pyrophosphate analogues that contain two phosphonate groups along with R¹ and R² variable side chains all bound to a central carbon. Bisphosphonates localize to bone due to two phosphonate groups bonding to hydroxyapatite crystals and the variable group at the R¹ position (which may be a hydroxyl group that has a higher affinity for calcium than a halogen). After bone localization, the antiresorptive potency of the bisphosphonate is also dependent on the three-dimensional R² side chain conformation, the overall chemical structure, and both phosphonate groups (Russell, 2007).

Two subclasses of bisphosphonates with differing chemical structures and functions are: 1) nonaminobisphosphonates and 2) aminobisphosphonates, which contain a nitrogen-containing side chain at the R² position such as alendronate, ibandronate, pamidronate, risedronate and zoledronate that dramatically enhances potency. As these compounds lack an amino-nitrogen atom, nonaminobisphosphonates, such as etidronate, clodronate and tiludronate, are more similar to inorganic pyrophosphate. Hence, osteoclasts “metabolically incorporate” nonaminobisphosphonates to form nonhydrolyzable adenosine triphosphate analogues, which in turn results in apoptosis of the osteoclast (Russell, 2007).

Despite this occurrence, the presence of the amino-nitrogen atom at the R² position transforms aminobisphosphonates into more potent antiresorptive compounds than nonaminobisphosphonates (up to 10000-fold more potent). The main function of aminobisphosphonates involves inhibition of a key component of the mevalonate pathway (cholesterol synthesis), specifically the enzyme farnesyl diphosphonate synthase. Disruption of cholesterol synthesis is deleterious to various cellular activities in the osteoclast, which arrests osteoclast activity and ultimately bone resorption (Woo et al., 2006; Russell, 2007). Bisphosphonates are currently widely used as first-line treatment and have become a cornerstone in the management of osteoporosis, Paget disease of the bone, and other metabolic bone diseases as well as in managing the skeletal complications of malignancy.

Currently, there is no standard and reliable test to diagnose, anticipate or predict MRONJ development to benefit patients. Treatment is at the discretion of the clinician, dependent on a number of factors including the stage of the condition and duration of bisphosphonate use (Woo et al., 2006; Ruggiero 2008). One treatment involves the use of antibiotics with adjunct application of an anti-microbial oral rinse, such as the standard 0.12% chlorhexidine rinse with antibiotics (Penicillin VK 500 mg) (Ruggiero, 2008). Various studies examining the use of chlorhexidine with MRONJ patients indicate that while it may only help stop MRONJ progression, it does not lead to assured MRONJ resolution in all patients (Estilo et al., 2008). Other suggested treatments include, but are not limited to: hyperbaric oxygen therapy, surgical debridement/resection, halting bisphosphonate therapy, and use of other anti-bacterial oral rinses (AAOMS, 2007; Khosla et al., 2007; Ruggiero, 2008). In general, treatments also include daily irrigation and antimicrobial rinses, antibiotics to control infection, surgical treatment and debridement to remove the necrotic bone, and fabricate a new prosthesis which should advised patients removed and thoroughly clean each nigh if applicable due to irritate to soft-tissues. The current goals for MRONJ treatment are: 1) to preserve the patient's quality of life (through MRONJ prevention, managing pain and secondary infection, and/or stopping progression of the condition) and 2) to enable oncology patients continued bisphosphonate use (AAOMS, 2007). Therefore, MRONJ is identified and diagnosed by the clinical definition after the onset and persistence of symptoms, through visual observation by the clinician and/or the use of medical imaging such as radiographs (Mariotti, 2008; Khosla et. al., 2007). Clinical staging of MRONJ has been proposed to characterize the progression from AAOMS, 2007). At Risk stage comprises of individuals that exhibit no symptoms but are receiving either intravenous or oral bisphosphonate for months to years. Stage 1 involves the initial appearance of necrotic bone on the jaw but lacks the presence of infection. Stage 2 includes both the presence of necrotic bone on the jaw and infection, with associated pain. Stage 3 involves necrotic bone on the jaw, infection, pain “and one or more of the following: pathologic fracture, extraoral fistula, or osteolysis extending to the inferior border” (Ruggiero, 2008).

Patients receive bisphosphonates as primary and secondary therapies for diseases relating to abnormal bone resorption. Such patient populations include: 1) individuals at high risk for developing and diagnosed with osteoporosis, 2) bone cancer and multiple myeloma patients, 3) cancer patients at high risk for bone metastases, and 4) patients with Paget's disease (Ruggiero and Drew, 2007). Bisphosphonate therapies may be taken long-term (months to years), as is the case for the prevention and treatment of osteoporosis, where these drugs may be taken for up to a decade or more. Furthermore, bisphosphonates for cancer treatments may also be indicated over months as well. Since an individual may receive bisphosphonates over many months or years, a need exists for a long-term MRONJ treatment and preventative that is safe and efficacious throughout the term of bisphosphonate therapy.

Since the pathophysiology of MRONJ is not well determined and there are no biomarkers to the development of the disease, the options for prevention are limited. One key prevention recommendation, however, is “to maintain good oral hygiene” and to receive a dental examination before beginning bisphosphonate therapy. Other recommended steps include having any needed invasive dental procedures prior to beginning a bisphosphonate regime and/or stopping bisphosphonate use prior to or during execution of the dental procedures (AAOMS, 2007; Khan, 2008). Be sure removable prostheses if applicable, fit well, comfortable, and removed and thoroughly cleaned each night. Refraining from smoking and minimizing alcohol consumption during bisphosphonate usage is also advised (Khan, 2008). MRONJ preventative strategies vary according to the type of bisphosphonate being administered (intravenous versus oral) and the duration of use (Khosla, et al., 2007).

The specific association between bisphosphonates and MRONJ development is not known nor has a direct causal link between bisphosphonate usage and the onset of MRONJ disease been definitively established (ADA Council on Scientific Affairs Expert Panel ONJ, 2008; Mariotti, 2008). Certain factors however are believed to potentially increase an individual's risk for developing MRONJ, such as: “1) history of dento-alveolar trauma, 2) duration of bisphosphonate exposure,” and 3) the type and route of bisphosphonate administration such as oral versus intravenous (Ruggiero and Drew, 2007) with some other factors such as radiation therapy of the facial area, trauma (osteotomy of the jaw bone or during intubation), viral infection (herpes zoster or HIV), fungal infection with Aspergillus (Z. Yehuda, 2012), circulatory insufficiency, local application of chemical agents in dental treatment, inhaling cocaine, and osteomyelitis (Woo et al, 2009). Thus, MRONJ prevalence and incidence are currently being investigated taking these factors into account. For example, one inquiry evaluating MRONJ prevalence among oral alendronate users reported an MRONJ prevalence rate of 4% in 208 patients (Sedghizadeh, et al., 2009). It is also noted that aminobisphosphonates, such as pamidronate and zoledronic acid, are the bisphosphonate subclass most often associated with MRONJ occurrence. One study stated that 94% of the reported ONJ patients had received zoledronic acid, pamidronate or a combination of the two drugs (Woo et al., 2006).

The pathophysiology of MRONJ has not fully elucidated and currently still under investigation. Many journal articles and reviews have been published evaluating the possible pathophysiology of this condition. One hypothesis suggested by Woo et al. (2006) is that bisphosphonate-associated osteonecrosis of the jaws results from marked suppression of bone metabolism that results in accumulation of physiologic microdamage in the jawbones, compromising biomechanical properties. Trauma and infection increase demand for osseous repair that exceeds the capacity of the hypodynamic bone, resulting in localized bone necrosis. The antiangiogenic property of bisphosphonates and other medications and the presence of other comorbid factors may promote the risk for or persistence and progression of this condition.”

Along with the suppression of bone metabolism due to bisphosphonates, microbial biofilms are also hypothesized to be involved in MRONJ. Biofilms are believed to be a source of microbial infection that can lead to development or increase progression of MRONJ. According to Donlan and Costerton, “a biofilm is a microbial derived sessile community characterized by cells that are irreversibly attached to a substratum, interface or to each other, are embedded in a matrix of extracellular polymeric substances that they have produced, and exhibit an altered phenotype with respect to growth rate and gene transcription” (Donlan and Costerton, 2002). Multispecies microbial biofilms were recently identified in bone specimens from MRONJ lesions of four MRONJ patients (Sedghizadeh et al., 2008). Specific pathogens classified in these MRONJ biofilms were from genera such as: Fusobacterium, Bacillus, Actinomyces, Staphylococcus, treponemes, and Candida, among others. The bacteria ranged from grain-positive and grain-negative organisms and included aerobes, although anaerobes and facultative anaerobes dominated. Known morphotypes of the Candida species were also apparent in the MRONJ biofilm of all four subjects and co-aggregation with bacteria also was observed. Sedghizadeh et al. further observed the absence of eukaryotic cells and the presence of microorganisms in the bone resorption pits of osteonecrotic bone specimens, indicating that microorganisms possibly directly contribute to bone resorption as well. Taken together, the presence of biofilms in MRONJ may potentially contribute to development and progression (Sedghizadeh et al., 2008). The ADA Council on Scientific Affairs Expert Panel on MRONJ (2008) mentioned a second hypothesis that the bisphosphonate compounds themselves are toxic to the tissues vulnerable to MRONJ development. Preliminary in vitro evidence on oral mucosal cells (human gingival fibroblasts and keratinocytes) indicates that direct application of zoledronic acid has a deleterious effect on the life of these cells (Scheper et al., 2008). As a result, bisphosphonates can also be a factor in MRONJ development when direct toxicity to oral mucosa cells.

The term stannous fluoride (SnF₂) is widely used in the dentistry over 40 years. Stannous fluoride has antiviral, bactericides and bacteriostatic properties, and anti-fungal effect. It has been found that stannous fluoride contains anesthetic, analgesic, and/or antibiotic therapeutic properties. Stannous fluoride exerts antibacterial effects by two modes of action. First, stannous fluoride exerts a killing effect on bacteria (bactericidal action). This is probably due to non-specific interaction with the bacterial membrane that causes membrane disruption. The result is leakage of cellular components that leads to cell lysis and death. The second, and more important, mode of antibacterial activity is through stannous fluoride's inhibition of metabolic enzymes. The inhibition of metabolic activity affects bacteria in a number of ways, including, 1) reduction of bacterial growth, 2) prevention of bacterial adhesion to oral surfaces (e.g., enamel, exposed dentin), 3) reduction in bacterial byproducts that boost the inflammatory response leading to gingivitis.

Stannous fluoride's inhibitory effect on bacteria is related to its inhibition of bacterial glycolysis, an energy making process whereby metabolic enzymes break down carbohydrates. Stannous fluoride has shown that significantly reduces metabolic toxins produced by bacteria in plaque biofilm. (Ramji N et al, Mankodi S et al) There are more than 750 bacteria species existing as mixed biofilm community in the oral cavity such as Actinomyces, Bacillus, Mycobacteria, Pseudomonas, Sphingomonas, Staphylococcus, Streptococcus, Enterococcus, Candida albicans, Aspergillus, Haemophilus influenzae, alpha-hemolytic streptococci, Lactobacillus, Enterobacter, and Klebsiella pneumoniae. The most commonly bacteria present of bacteria biofilm which presents at MRONJ sites is Actinomyces which has been demonstrated by histomorphometric and histological studies.

### SUMMARY OF THE INVENTION

The composition is comprised of a 0.005%-1.5% (w/v) stannous fluoride and may take the form of a gel which can facilitate the treating and healing of osteonecrosis of the jaw including but not limited to necrotic bone lesions, inflammation and infection. The related methodology includes the application of the composition in the oral cavity affected by osteonecrosis of the jaw to produce an antimicrobial effect, sanitize, debride, and penetrate, eliminate and control osteonecrosis of the jaw biofilms associated with osteonecrosis of the jaw which facilitates the healing of osteonecrosis of the jaw symptoms and prevents the development of osteonecrosis of the jaw. The application may be daily or continuously as a medication-related osteonecrosis of the jaw (MRONJ) treatment regimen or as an osteonecrosis of the jaw prevention regimen.

Another object of the present invention is to facilitate healing of Medication-related osteonecrosis of the jaw (MRONJ) symptoms by the application of the composition to the oral cavity and other osteonecrosis of the jaw affected tissues. Still another object of the present invention is for the composition to produce antimicrobial, sanitizing, and debriding effects on tissues affected by or susceptible to osteonecrosis of the jaw and facilitate healing of medication-related osteonecrosis of the jaw (MRONJ) symptoms and prevent of osteonecrosis of the jaw.

A further object of the present invention is for the composition to penetrate, eliminate and control biofilms that form over tissues that are affected lesion by or susceptible to medication-related osteonecrosis of the jaw (MRONJ). Yet a further object of the present invention is to apply the composition on a daily or continuous basis to the oral cavity affected by medication-related osteonecrosis of the jaw (MRONJ).

A still further object of the present invention is to apply the composition upon the onset of osteonecrosis of the jaw symptoms (including but not limited to, necrotic bone lesions, inflammation, and infection) and/or prior to or throughout bisphosphonate use.

These and other objects and specific embodiments of the present invention will become apparent to those skilled in the art as the description thereof proceeds.

### DESCRIPTION OF THE PREFERRED EMBODIMENT

### Potential Mechanism of Action of the Invention

The pathophysiology of osteonecrosis of the jaws and medication-related osteonecrosis of the jaw (MRONJ) are unknown. Therefore, any suggested mechanism of action for the stannous fluoride composition is hypothesized on the following: 1) published clinical experimental evidence investigating MRONJ, 2) previous work describing the treatment of malodor, gingivitis, and periodontitis, 3) previous work describing the properties of stannous fluoride.

A possible means for treating and treatment of MRONJ is based on the hypothesis that bisphosphonates are anti-angiogenic, depriving tissues of nutrients, leading to the presence of microbes which in turn impedes the healing process of tissues existing in a depressed metabolic state (Adornato et al., 2007; Woo et al., 2006). Furthermore, the mode of action is also based on the hypothesis that biofilms involved with MRONJ are associated with conditions that may lead to MRONJ development and progression (Sedghizadeh et. al., 2008). It has shown that the present invention produces an antimicrobial effect, sanitizes, and debrides MRONJ based on previous work which investigated the microbicidal activity, sanitizing, and debriding properties of stannous fluoride. It is believed that the present invention may penetrate, eliminate, and control biofilms (biofilms which may contain microorganism species such as Candida or Streptococcus) that form over ONJ lesions or tissues susceptible to ONJ formation. It has found that Bron Gel has antifungal effect.(Lin C, manuscript on preparation) This assumption is based on work investigating the characteristics of biofilms on MRONJ lesions, and the ability of a stannous fluoride to decontaminate biofilms as well as the microbicidal and bacteriostatic properties of stannous fluoride. It is further thought that the present invention results in less extensive toxic effects on MRONJ-associated cell types, associated with wound healing, compared with alternative oral rinses currently used to prevent and treat MRONJ, thereby facilitating healing of MRONJ or preventing MRONJ development (Wirthlin et. al., 2006; Patel et. al., 2006; Gianelli et. al., 2008; Nishikori, et. al., 2008). The ability of a stannous fluoride to act as an antimicrobial, sanitize, debride, penetrate, eliminate and control MRONJ biofilms, and reduce the toxicity to cells that are critical to wound healing and fistula, the present invention provides an antiseptic environment that facilitates healing of MRONJ symptoms and results in the subsequent resolution of MRONJ symptoms.

### EXAMPLE 1

### Stannous Fluoride Potential Efficacy as an MRONJ Treatment (Chun Nan Lin, 2017)

Dr. Chun Nan Lin has observed that the use of stannous fluoride gel resulted in the resolution of MRONJ lesions and heal without any complication. Five subjects were observed to have MRONJ lesions and associated symptoms such as fistula on the jaws. One MRONJ patient was treated with mechanical intervention, 0.12% Chlorhexidine oral rinse, and antibiotic therapy. The remaining four MRONJ patients were treated with Stannous fluoride onto the MRONJ lesions.

The patient treated by antibiotic therapy had been receiving intravenous zoledronate (Zometa, Novartis; East Hanover, N.J.) due to multiple myeloma The patient presented a bone lesion that was smoothed and then treated with an antibiotic (Augmentin, SmithKline Beecham Pharmaceuticals: Philadelphia, Pa.). Observations continued for a little over a year, and the lesion continued to increase in size and did not heal with this antibiotic regimen.

Three patients presenting MRONJ lesions had received or were currently receiving long-term alendronate therapy (Fosamax, Merck & Co Inc.; Whitehouse Station, N.J.). Two of these patients underwent tooth extractions either prior to or while on the alendronate therapy. All three patients were instructed to inject stannous fluoride gel into the MRONJ lesion, two to three times a day. The patients experienced complete healing of the MRONJ lesions and affected tissue within two to three months. An example of healing was shown by gingival healing, fistula healing, and closure of the diseased area with no bone exposure. The bone of MRONJ lesion was complete heal like before.

### EXAMPLE 2

### Antimicrobial, Sanitizing, and Debriding Properties of Stannous Fluoride Gel

Chun Nan Lin, D.D.S., M.S. (2005) performed an experiment to investigate a potential mechanism of action of stannous fluoride gel against C. albicans. This study examined the ability of stannous fluoride gel to inhibit the growth of C. albicans. C. albicans was exposed to varying concentrations of stannous fluoride gel, and plasma membrane damage was assessed over a time or dose dependent evaluation.

### EXAMPLE 3

### Chlorhexidine Inability to Resolve MRONJ and Treat MRONJ

A retrospective study was conducted of 4,835 intravenous bisphosphonate users treated at Memorial Sloan-Kettering Cancer Center (MSKCC). Memorial Sloan-Kettering Cancer Center Dental Service received 310 of these patients referred between Jan. 1, 1996 and Jan. 31, 2006. Thirty-five of these individuals were diagnosed with MRONJ either at the initial dental evaluation or a subsequent dental visit and all were treated with 0.12% chlorhexidine as part of the MRONJ treatment regimen. Eleven patients did not reach the end point of the study. Out of the remaining 24 patients treated with chlorhexidine, only 3 patients experienced MRONJ resolution, while the other 21 patients either experienced MRONJ progression (13 patients) or experienced no change in ONJ status (8 patients) (Estilo et. al., 2008).

### EXAMPLE 4

### Evidence of Anti-Angiogenic Effects of Bisphosphonate

Research evidence cited by Adornato et al indicates certain bisphosphonates negatively affect vascularity due to anti-angiogenic effects on bone. Specifically, pamidronate was shown to reduce bone vascularity in rats. Both zoledronic acid and pamidronate displayed the ability to inhibit the new formation of capillaries from blood vessels (capillary neoangiogenesis). (Adornato et al., 2007) The possible effects these drugs have on angiogenesis and bone vascularity indicate a potential explanation of MRONJ pathophysiology, as both drugs are known to be associated with MRONJ (Woo et. al., 2006).

### EXAMPLE 5

### Evidence of Biofilm and Microbes in MRONJ and the Ability of Stannous Fluoride Gel to Penetrate and Decontaminate Biofilms

Microbial biofilms have been detected in osteonecrotic regions of the jaw and some microbial genera that comprise these biofilms have been identified by Sedghizadeh et al. (2008). Of the microbial genera identified in MRONJ biofilms, similar genera have also been detected in biofilms such as Actinomyces spp, Bacillus spp, Staphylococcus, and Streptococcus. (Wirthlin et al., 2003; Sedghizadeh et al., 2008) Based on stannous fluoride composition, stannous fluoride is relatively stable in the glycerin gels, but once applied into the oral cavity or in the bioform model, stannous fluoride may react with water and precipitate from solution to form Sn(OH)₂ or oxidize with oxygen and form Sn4⁺which have antimicrobial role. (Tinaoff N, 1995) Hence, despite all of above evidences for how stannous fluoride can facilitate resolution of MRONJ, it is still not clear as to how or whether biofilms contribute to MRONJ development and/or progression.

### REFERENCES

### Title: Composition and method for treating and healing Medication-Related Osteonecrosis of the Jaw (MRONJ)

### Patents Cited in This Cited by

| | | Application | Publication |
|---|---|---|---|
| Title | Assignee | Date | Date |
| A Method and composition | Ratcliff Perry | 1984 Jul. 30 | 1987 Sep. 29 |
| for prevention and treatment | | | |
| of oral disease | | | |
| A Method and composition | Ratcliff Perry | 1984 Jul. 30 | 1988 Nov. 29 |
| for prevention and treatment | | | |
| of oral disease | | | |
| A Method for preventing | Ratcliff Perry | 1984 Jul. 30 | 1989 Dec. 12 |
| Periodontitis | | | |
| Oral health preparation | Micropure, Inc. | 1990 Nov. 20 | 1993 Apr. 6 |
| and method | | | |
| Oral health preparation | Micropure, Inc. | 1990 Nov. 20 | 1994 Sep. 20 |
| and method | | | |
| Composition for treatment | Ratcliff Perry | 1993 Jul. 6 | 1996 Feb. 6 |
| of abnormal conditions of | | | |
| the epithelium of bodily | | | |
| orifices | | | |
| Oral rinse and method of | Richter Jon L | 1994 Apr. 7 | 1995 Oct. 19 |
| treating halitosis | | | |
| Promoting whole body | The Procter & | 1998 Feb. 27 | 2005 Jan. 25 |
| Health | Gamble Company | | |
| Effects of Stannous Fluoride | Lin et al | 2005 | |
| on In Vitro growth of | | | |
| Candida Species | | | |

| | | |
|---|---|---|
| Antiresorptives and | Yamashita J, | 2012; 12(3 Suppl): 233-47. |
| osteonecrosis of the jaw | McCauley LK | |
| | J Evid Based Dent Pract | |

We claim:

1. A composition is a gel for treating and healing medication-related osteonecrosis of the jaw (MRONJ) with a non-invasive procedure, said composition comprising:
(a) an alcohol free composition for application to MRONJ tissues and lesions comprising a solution of a stannous salt having a concentration in the range of about 0.005% to about 1.5% weight/volume (w/v) selected from the group consists of stannous fluoride, stannous chloride, stannous salts;
(b) composition comprising the most non-ionic thicker, hydroxyethylcelluose, which having an antimicrobial effect against free-floating microorganisms and fungus that are present in lesions and tissues affected by MRONJ in the range of about 0.25% to about 0.45%;
(c) A composition can penetrating, controlling or eliminating biofilm that forms over tissues affected by or susceptible to MRONJ;
(d) A composition can producing an antimicrobial effect against microorganisms and fungus present in biofilm formed over tissues affected by or susceptible to MRONJ; and
(e) A composition can be non-invasively debriding oral tissue affected by MRONJ.

2. The composition as set forth in claim 1 wherein said composition is in the form of a gel.

3. The composition as set forth in claim 1 wherein the weight/volume (w/v) of the glycerin is>95%.

4. The composition as set forth in claim 1, wherein the diseased area has become closed with both of no bone exposure and no fistula.

5. A composition for penetrating, controlling and eliminating the biofilm associated with Medication-Related Osteonecrosis of the Jaw (MRONJ), said composition comprising:
an alcohol free composition for application to MRONJ tissues and lesions comprising a solution of a stannous fluoride gel having a concentration in the range of about 0.005% to about 1.5% weight/volume (w/v) selected from the group consisting of stannous fluoride, stannous chloride, and stannous salts.

6. The composition as set forth in claim 5 wherein the diseased area becomes closed with no bone exposure and no fistula.
